(19) United States
(12) Patent Application Publication
(10) Pub. No.: US 2025/0388966 A1
(43) Pub. Date: Dec. 25, 2025

(54) PRIMER COMBINATION AND KIT FOR IDENTIFYING GENETIC SEX OF STURGEON, AND USE THEREOF

(71) Applicant: CHINESE STURGEON RESEARCH INSTITUTE, CHINA THREE GORGES CORPORATION, Yichang (CN)

(72) Inventors: Yacheng Hu, Yichang (CN); Wei Jiang, Yichang (CN); Hejun Du, Yichang (CN); Xueqing Liu, Yichang (CN); Xin Zhu, Yichang (CN); Zhiyuan Li, Yichang (CN); Binzhong Wang, Yichang (CN); Yuanjin Yang, Yichang (CN); Kan Xiao, Yichang (CN); Chuan Wu, Yichang (CN); Huantao Qu, Yichang (CN)

(21) Appl. No.: 18/939,623

(22) Filed: Nov. 7, 2024

Related U.S. Application Data
(63) Continuation of application No. PCT/CN2024/102117, filed on Jun. 27, 2024.

(30) Foreign Application Priority Data
Jun. 21, 2024 (CN) 202410815667.0

(51) Int. Cl. C12Q 1/6879

(52) U.S. Cl. CPC C12Q 1/6879

(57) ABSTRACT

The present application provides a primer combination and a kit for identifying the genetic sex of sturgeon, and a use thereof. Using two pairs of primers can not only identify the genetic sex of sturgeon in a simple and rapid manner, but also effectively avoid the experimental errors, thereby improving the accuracy of sturgeon sex identification. At the same time, the present application is not limited by the age of sturgeon and would not cause significant damage to the sturgeon. It also has good universality and may provide assistance for sex-controlled breeding of sturgeon and protection and utilization of germplasm resources.

### CROSS-REFERENCE TO RELATED APPLICATIONS

The present application is a continuation of International Application No. PCT/CN2024/102117, filed on Jun. 27, 2024, which claims priority to Chinese Patent Application No. 202410815667.0, entitled “PRIMER COMBINATION AND KIT FOR IDENTIFYING GENETIC SEX OF STURGEON, AND USE THEREOF”, and filed with the China National Intellectual Property Administration on Jun. 21, 2024. The disclosures of the aforementioned applications are hereby incorporated by reference in their entireties.

### TECHNICAL FIELD

The present application relates to molecular biotechnology, particularly to a primer combination and a kit for identifying genetic sex of sturgeon, and a use thereof.

### SEQUENCE LISTING

A sequence listing is submitted electronically herewith and is incorporated herein by reference (filename: CNPCTP2408351-seq12.xml, date created: Jan. 7, 2025, file size 8 kilobytes). The Sequence Listing submitted is part of the specification and is herein incorporated by reference in its entirety.

### BACKGROUND

Sturgeon is the abbreviation of Acipenseriformes, and is the only existing large chondrostei fish in the class of Osteichthyes. It has a long sexual maturity age, spawns and reproduces in rivers, and most species have the habit of migrating between rivers and seas. There are 27 species existing worldwide for sturgeon, which is of enormous academic significance and economic value. However, the intensification of human activities and overfishing over the past century have led to a sharp decline in all sturgeon resources, and various species are currently in varying degrees of endangered status, which has attracted widespread attention from society.

Sturgeon does not have obvious external secondary sexual characteristics such as sex chromatic organ, so it is difficult to distinguish male sturgeon from immature female sturgeon in non-spawning areas. In the spawning area, semen may generally be squeezed out from the genital pore of mature male sturgeon, while female sturgeon generally has an expanded abdomen and a thicker waistline. However, a small-size female sturgeon that is about to spawn for the first time is often confused with the male sturgeon without semen and the immature female sturgeon. According to the external morphology of the genital pore of sturgeon, male and female sturgeons can be identified. Observation on wild adults of Acipenser transmontanus of North American, Acipenser medirostris, Acipenser oxyrinchus, and Acipenser brevirostrum revealed that the genital pore of female sturgeon generally forms an “O” shape, while the genital pore of male sturgeon generally forms a “Y” shape. The accuracy of this identification method may reach 82% in live and fresh specimens, but only 29% in dead specimens. Some scholars have identified the gender of Acipenser gueldenstaedti by measuring measurable head traits, with an accuracy rate of 90% or more. The most straightforward method of sex identification for sturgeon with gonadal development above stage II is to distinguish an ovary from a testis through abdominal surgical techniques. However, the above-mentioned sex identification methods for sturgeon can only identify sturgeon that has developed to a certain age, but cannot be used for sex identification of juvenile sturgeon; and some methods of sex identification may cause significant damage to sturgeon, such as abdominal surgery techniques. It has been reported that use of the female-specific DNA fragment of sturgeon in the sex identification method of sturgeon. During the operation of this method, unsuccessful extraction of DNA, wrong addition of drugs or omission of drugs will misidentify the sturgeon to be identified as a male sturgeon, resulting in wrong sex identification of sturgeon.

Exploring the regulatory mechanism of sex differentiation in sturgeons and establishing new technologies for breeding all female or high-ratio female sturgeons is of important theoretical and practical significance for the protection and development and utilization of its germplasm resources. The development of sex-specific molecular markers helps to accurately and quickly obtain phenotypes and determine gender, so as to carry out the work such as artificial breeding and selection. It also helps to conduct research on sex-controlled breeding techniques in commercial production, and further establish a mono-sexual system. Therefore, developing a molecular marker that accurately identifies the sex of sturgeon has great production and applied value in sturgeon artificial breeding.

### SUMMARY

The present application provides a primer combination and a kit for identifying genetic sex of sturgeon, and a use thereof, which improves the accuracy of sturgeon sex identification and the sex identification may be performed throughout the entire life cycle of sturgeon.

The present application also provides a method for identifying genetic sex of sturgeon, using the above-mentioned primer combination or the above-mentioned kit, which has simple operation and accurate result, and the identification may be performed throughout the entire life cycle of sturgeon.

The present application provides a primer combination for identifying genetic sex of sturgeon, including a first primer pair and a second primer pair. The first primer pair includes a first primer and a second primer, and a nucleotide sequence of the first primer is shown in SEQ ID NO: 1 and a nucleotide sequence of the second primer is shown in SEQ ID NO: 2. The second primer pair includes a third primer and a fourth primer, and a nucleotide sequence of the third primer is shown in SEQ ID NO: 3 and a nucleotide sequence of the fourth primer is shown in SEQ ID NO: 4.

For the primer combination as described above, the first primer pair is used for amplifying a specific DNA fragment of a female sturgeon, and the specific DNA fragment of the female sturgeon has a nucleotide sequence shown in SEQ ID NO: 5 and a fragment size of 473 bp. The second primer pair is used for amplifying a DNA fragment shared by the female sturgeon and a male sturgeon, and the DNA fragment shared by the female sturgeon and the male sturgeon has a nucleotide sequence shown in SEQ ID NO: 6 and a fragment size of 717 bp.

For the primer combination as described above, the sturgeon includes at least one of Acipenser sinensis, Acipenser dabryanus, Acipenser ruthenus, Acipenser schrenckii, Acipenser schrenckii♂×Huso dauricus♀, Acipenser schrenckii♂×Acipenser baerii♀, Acipenser baerii, Huso dauricus, Acipenser gueldenstaedti, Acipenser stellatus, Huso huso, Acipenser fulvescens, Acipenser nudiventris, Acipenser persicus, Acipenser transmontanus, or Acipenser medirostris.

The present application provides a kit for identifying genetic sex of sturgeon, including the primer combination described above.

For the kit as described above, the kit further includes at least one of water, PCR buffer, dNTPs, DNA polymerase, or loading buffer.

The present application provides a use of the above-mentioned primer combination or the above-mentioned kit in identifying genetic sex of sturgeon.

The present application further provides a method for identifying genetic sex of sturgeon, including the following steps of extracting DNA of a sturgeon sample to be detected; performing a PCR amplification using the above-mentioned primer combination or the above-mentioned kit with the DNA of the sturgeon sample to be detected as a template to obtain a PCR amplification product; performing an agarose gel electrophoresis detection on the PCR amplification product, and identifying the genetic sex of sturgeon as female when two bands of 473 bp and 717 bp appear in a detection result and identifying the genetic sex of sturgeon as male when one band of 717 bp appears in the detection result.

For the method as described above, the sturgeon sample to be detected includes at least one of fin strip of the sturgeon or mucus of the sturgeon.

For the method as described above, in the PCR amplification, a PCR reaction system includes 3-5 μL of the DNA template, 0.5-1.5 μL of the first primer, 0.5-1.5 μL of the second primer, 0.25-0.75 μL of the third primer, 0.25-0.75 μL of the fourth primer, 10-15 μL of 2×Taq Master Mix, and 4-6 μL of ddH2O.

For the method as described above, in the PCR amplification, a PCR amplification program includes pre-denaturation at 93-95° C. for 3 minutes; denaturation at 93-95° C. for 10 seconds, renaturation at 54-56° C. for 10 seconds, and extension at 72° C. for 19 seconds, for a total of 35 cycles; and finally extension at 72° C. for 10 minutes.

The primer combination of the present application provides two pairs of primers, which can avoid experimental errors caused by unsuccessful DNA extraction, wrong addition of drugs or omission of drugs, thereby improving the accuracy of sturgeon sex identification. At the same time, the primer combination of the present application is not limited by the age of sturgeon and may be used for sex identification throughout the entire life cycle of sturgeon. The present application provides assistance for sex-controlled breeding of sturgeon and protection and utilization of germplasm resources, as well as technical support for biodiversity research and the great conservation work of Yangtze River.

### BRIEF DESCRIPTION OF DRAWINGS

FIG. 1 is an electrophoretogram for genetic sex identification of Acipenser sinensis in Embodiment 1.

FIG. 2 is an electrophoretogram for genetic sex identification of Acipenser dabryanus in Embodiment 2.

FIG. 3 is an electrophoretogram for genetic sex identification of Acipenser ruthenus in Embodiment 3.

FIG. 4 is an electrophoretogram for genetic sex identification of Acipenser schrenckii in Embodiment 4.

FIG. 5 is an electrophoretogram for genetic sex identification of Acipenser schrenckii♂×Huso dauricus♀ in Embodiment 5.

FIG. 6 is an electrophoretogram for genetic sex identification of Acipenser schrenckii♂×Acipenser baeriio in Embodiment 6.

FIG. 7 is an electrophoretogram for genetic sex identification of Acipenser baerii in Embodiment 7.

FIG. 8 is an electrophoretogram for genetic sex identification of Huso dauricus in Embodiment 8.

FIG. 9 is an electrophoretogram for genetic sex identification of Acipenser gueldenstaedti in Embodiment 9.

FIG. 10 is an electrophoretogram for genetic sex identification of Acipenser stellatus in Embodiment 10.

FIG. 11 is an electrophoretogram for genetic sex identification of Huso huso in Embodiment 11.

FIG. 12 is an electrophoretogram for genetic sex identification of Acipenser fulvescens in Embodiment 12.

FIG. 13 is an electrophoretogram for genetic sex identification of Acipenser nudiventris in Embodiment 13.

FIG. 14 is an electrophoretogram for genetic sex identification of Acipenser persicus in Embodiment 14.

FIG. 15 is an electrophoretogram for genetic sex identification of Acipenser transmontanus in Embodiment 15.

FIG. 16 is an electrophoretogram for genetic sex identification of Acipenser medirostris in Embodiment 16.

### DESCRIPTION OF EMBODIMENTS

In order to enable the person skilled in the art to better understand the solutions of the present application, further detailed explanations of the present application are provided below. The embodiments given below are only for the description of the principles and features of the present application; and the examples given are only for illustrating the present application, instead of limiting the scope of the present application. Based on the embodiments in the present application, all other embodiments obtained by the person skilled in the art without creative work fall within the protection scope of the present application.

A first aspect of the present application provides a primer combination for identifying genetic sex of sturgeon, including a first primer pair and a second primer pair. The first primer pair includes a first primer and a second primer. A nucleotide sequence of the first primer is as shown in SEQ ID NO: 1, and a nucleotide sequence of the second primer is as shown in SEQ ID NO: 2. The second primer pair includes a third primer and a fourth primer. A nucleotide sequence of the third primer is as shown in SEQ ID NO: 3, and a nucleotide sequence of the fourth primer is as shown in SEQ ID NO: 4.

The primer combination for identifying the genetic sex of sturgeon includes a first primer pair and a second primer pair. In the first primer pair of the present application, the first primer is an upstream primer and the second primer is a downstream primer. In the second primer pair of the present application, the third primer is an upstream primer and the fourth primer is a downstream primer.

The present application has obtained a primer combination for identifying the genetic sex of sturgeon based on the genomic DNA of sturgeon. When designing the primer combination, the present application not only considers how to identify the genetic sex of sturgeon in a simple and rapid manner, but also considers how to avoid experimental errors as the core factor, thus obtaining the primer combination including the first primer pair and the second primer pair.

Where, the first primer pair is intended to identify whether the genetic sex of sturgeon is female or not in a simple and rapid manner, and the second primer pair is intended to avoid experimental errors caused by unsuccessful DNA extraction, wrong addition of drugs or omission of drugs. When a DNA fragment shared by female sturgeon and male sturgeon is obtained by amplification using the second primer pair, it is proved that the experimental operation is correct or the experimental materials are normal and there is no situation such as unsuccessful DNA extraction, wrong addition of drugs or omission of drugs. At this point, the accuracy of the amplified results by the first primer pair is higher.

Moreover, in order to reduce the impact of DNA fragment deletion due to severe DNA degradation on the identification of sturgeon genetic sex, primer pairs as shown in SEQ ID NO: 1 to SEQ ID NO: 4 are designed. The amplified fragments obtained based on these primer pairs are not only located in the same chromosome, but also have a distance of no more than 20 kbp between them. Therefore, even if severe DNA degradation occurs, it will not affect the identification results.

Therefore, the primer combination of the present application not only has the ability to identify the genetic sex of sturgeon in a simple and rapid manner, but also has the effect of avoiding experimental errors and thus improving the accuracy of sturgeon sex identification.

Further, the first primer pair is used for amplifying a specific DNA fragment of female sturgeon, which has a nucleotide sequence shown in SEQ ID NO: 5 and a fragment size of 473 bp. The second primer pair is used for amplifying a DNA fragment shared by female sturgeon and male sturgeon, which has a nucleotide sequence shown in SEQ ID NO: 6 and a fragment size of 717 bp.

In order to ensure the accuracy of the amplification results, the amplified fragments are limited in the present application. When the amplified results do not meet the above limitations, it indicates that there may be an error in the experimental operations or there may be a problem with the experimental materials. Therefore, further experiments can be conducted to ensure the accuracy of the results.

The primer combination is not limited in the present application and may be used for the genetic sex identification of various sturgeons, such as Acipenser sinensis, Acipenser dabryanus, Acipenser ruthenus, Acipenser schrenckii, Acipenser schrenckii♂×Huso dauricus♀, Acipenser schrenckii♂×Acipenser baerii♀, Acipenser baerii, Huso dauricus, Acipenser gueldenstaedti, Acipenser stellatus, Huso huso, Acipenser fulvescens, Acipenser nudiventris, Acipenser persicus, Acipenser transmontanus, and Acipenser medirostris.

Where, Acipenser schrenckii♂×Huso dauricus♀ refers to a first-generation offspring of hybridization between Acipenser schrenckii and Huso dauricus. In one embodiment of the present application, the Acipenser schrenckii♂×Huso dauricus♀ used is the first-generation offspring of hybridization with the Acipenser schrenckii as a male parent and the Huso dauricus as a female parent. Acipenser schrenckii♂×Acipenser baerii♀ is the first-generation offspring of hybridization between Acipenser baerii and Acipenser schrenckii. In one embodiment of the present application, the Acipenser schrenckii♂×Acipenser baerii♀ used is the first-generation offspring of hybridization with the Acipenser schrenckii as the male parent and Acipenser baerii as the female parent.

A second aspect of the present application provides a kit for identifying genetic sex of sturgeon, including the primer combination provided in the first aspect of the present application.

Due to the inclusion of the primer combination provided in the first aspect of the present application, the kit also has the ability of identifying the genetic sex of sturgeon in a simple and rapid manner and has the effect of avoiding experimental errors and thus improving the accuracy of sturgeon sex identification.

In a specific implementation, the kit further includes at least one of water, PCR buffer, dNTPs, DNA polymerase, and loading buffer.

Where, water may provide the ionic environment and water molecule medium required for PCR reaction, enabling smooth interactions between various molecules in the reaction, and it may also help maintain the temperature stability of the reaction system, ensuring the reliability and accuracy of the reaction. PCR buffer is able to adjust the pH value of the reaction system, so that the operation environment of Taq DNA polymerase remains alkaline, and the Mg2+ in the PCR buffer can also affect the specificity of the reaction and the yield of amplified fragments. The dNTPs refer to four kinds of free deoxyribonucleoside triphosphates, which are essential raw materials for PCR amplification. DNA polymerase refers to a type of enzyme that catalyzes the polymerization of substrate dNTP molecules to form offspring DNA using parental DNA as a template. Generally, DNA polymerase in PCR technology is also thermally stable. The loading buffer may increase the sample density and prevent the sample from floating out of the sample well. Further, the indicator contained in the loading buffer may show the progress of electrophoresis. Some loading buffers also contain sodium dodecyl sulfate, which may promote the denaturation of DNA polymerase and prevent residual DNA polymerase from binding to DNA double strands and thus affecting the electrophoresis migration rate.

A third aspect of the present application provides a use of the above-mentioned primer combination or the above-mentioned kit in identifying genetic sex of sturgeon.

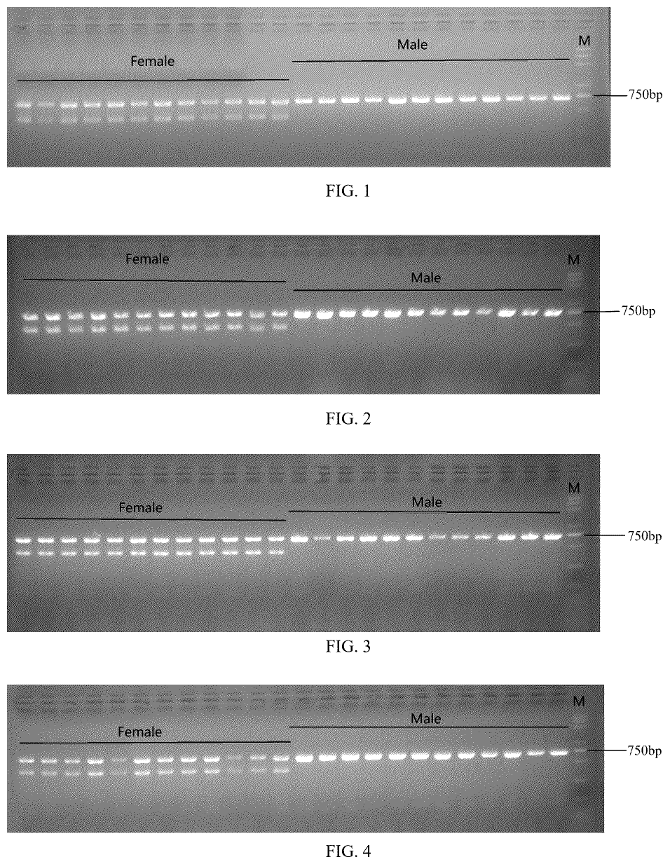

In a specific implementation, FIG. 1 shows the electrophoresis detection result of genetic sex identification of Acipenser sinensis provided in an embodiment of the present application. As shown in FIG. 1, when two bands of 473 bp and 717 bp appear in the detection result, the genetic sex of sturgeon is female; when one band of 717 bp appears in the detection result, the genetic sex of sturgeon is male.

A fourth aspect of the present application provides a method for identifying genetic sex of sturgeon, including the following steps: extracting DNA of a sturgeon sample to be detected; performing a PCR amplification using the above-mentioned primer combination or the above-mentioned kit with the DNA of the sturgeon sample to be detected as a template to obtain a PCR amplification product; performing an agarose gel electrophoresis detection on the PCR amplification product, and identifying the genetic sex of sturgeon as a female when two bands of 473 bp and 717 bp appear in the detection result and identifying the genetic sex of sturgeon as a male when one band of 717 bp appears in the detection result.

According to the primer combination provided in the first aspect of the present application, the DNA of the sturgeon sample to be detected is subjected to a PCR detection and an agarose gel electrophoresis detection, and the genetic sex of the sturgeon sample to be detected may be identified according to the number and size of the bands detected, that is, the gender of the sturgeon sample to be detected may be identified as female or male. The method for identifying genetic sex of sturgeon provided in the present application only requires the extraction of trace amount of DNA of the sturgeon to be detected, which does not cause significant impact or damage to the sturgeon, nor does it be limited by the age of the sturgeon, and the identification of genetic sex may be carried out throughout the entire life cycle of the sturgeon.

Specifically, when the identification is carried out in accordance with the steps described above, the sturgeon sample to be detected includes at least one of fin strip of the sturgeon or mucus of the sturgeon, where the mucus may specifically be the mucus on the body surface of sturgeon. Extraction of genomic DNA of the sturgeon sample to be detected may be achieved using conventional techniques in the art, such as CTAB (Hexadecyltrimethyl Ammonium Bromide) method.

Further, in the PCR amplification, a PCR reaction system includes 3-5 μL of the DNA template, 0.5-1.5 μL of the first primer, 0.5-1.5 μL of the second primer, 0.25-0.75 μL of the third primer, 0.25-0.75 μL of the fourth primer, 10-15 μL of 2×Taq Master Mix, and 4-6 μL of ddH₂O.

In addition, when a PCR amplification program includes pre-denaturation at 93-95° C. for 3 minutes; denaturation at 93-95° C. for 10 seconds, renaturation at 54-56° C. for 10 seconds, and extension at 72° C. for 19 seconds, for a total of 35 cycles; and finally extension at 72° C. for 10 minutes, it helps to improve the specificity of PCR reaction, thereby enhancing the accuracy of genetic sex identification in sturgeon.

Finally, the PCR amplification products are detected according to the conventional agarose gel electrophoresis detection method in the art. The genetic sex of the sturgeon sample to be detected may be identified based on the difference in number and size of bands shown by agarose gel electrophoresis.

Hereinafter, the technical solutions of the present application will be further explained and illustrated with specific embodiments.

In the following embodiments, the agarose gel electrophoresis is uniformly performed using a DNA molecular weight standard (DNA Marker, abbreviated as M) of DL5000, with electrophoresis bands representing 5000 bp, 3000 bp, 2000 bp, 1000 bp, 750 bp, 500 bp, 250 bp and 100 bp respectively from top to bottom. For the remaining experimental methods without specific conditions indicated, they are usually conducted under conventional conditions or according to the conditions recommended by the manufacturer. The reagents used, unless otherwise specified, are commercially available or publicly available.

The primer sequences involved in the following embodiments are shown in Table 1.

TABLE 1

| Primer Name | Sequence Name | Sequence (5′-3′) |
|---|---|---|
| XYJD15F | SEQ ID NO: 1 | TTCGGGCAGGACAACT |
| XYJD15R | SEQ ID NO: 2 | AACAATCGGGCAACCA |
| XYXY38F | SEQ ID NO: 3 | GGATTGAGGAGGATGTGC |
| XYXY38R | SEQ ID NO: 4 | GTGAAATAGGGGACTGGC |

### Embodiment 1: Identification of Genetic Sex of Acipenser sinensis

After the genetic sex of Acipenser sinensis was determined through spawning and sperm squeezing, fin strips were taken from 12 female Acipenser sinensis and 12 male Acipenser sinensis, where the Acipenser sinensis were from Chinese Sturgeon Research Institute, China Three Gorges Corporation. DNA samples were prepared by extracting DNA from the fin strips of 24 Acipenser sinensis mentioned above using a DNA extraction kit. The integrity of DNA samples was identified by 1.5% agarose gel electrophoresis, and then the DNA samples were measured for OD values by ultraviolet spectrophotometer and diluted to 50 ng/μL.

Primer XYJD15F and Primer XYJD15R were designed based on the specific DNA fragment of female sturgeon, where specific DNA fragment of the female sturgeon has a nucleotide sequence as shown in SEQ ID NO: 5 and a fragment size of 473 bp. Primer XYXY38F and Primer XYXY38R were designed based on the shared DNA fragment between female and male sturgeons, where the shared DNA fragment between female sturgeon and male sturgeon has a nucleotide sequence as shown in SEQ ID NO: 6 and a fragment size of 717 bp.

The above-mentioned DNA samples were used as a template, and amplified by PCR technology to obtain PCR products. The PCR amplification system was a total of 25 μL in volume, including 4 μL of DNA template, 1 μL of XYJD15F, 1 μL of XYJD15R, 0.5 μL of XYXY38F, 0.5 μL of XYXY38R, 12.5 μL of 2×Taq Master Mix (With Dye), and 5.5 μL of ddH₂O. The PCR amplification program included pre-denaturation at 94° C. for 3 minutes; denaturation at 94° C. for 10 seconds, renaturation at 56° C. for 10 seconds, and extension at 72° C. for 19 seconds, for a total of 35 cycles; and finally, extension at 72° C. for 10 minutes, and storage at 4° C. PCR products were added into 3% agarose gel, with a voltage set to 120V for 25 minutes for electrophoresis, and the agarose gel after electrophoresis was analyzed using a gel imaging system.

As shown in FIG. 1, it can be seen that all 12 female Acipenser sinensis showed two bands, with sizes of 473 bp and 717 bp, respectively; all 12 male Acipenser sinensis showed one band with a size of 717 bp. The results of this experiment were consistent with the results of genetic sex identification of Acipenser sinensis obtained through spawning and sperm squeezing.

### Embodiment 2: Identification of Genetic Sex of Acipenser dabryanus

The present Embodiment 2 may refer to the steps of Embodiment 1, with the difference being that: after the genetic sex of Acipenser dabryanus was determined through spawning and sperm squeezing, fin strips from 12 female Acipenser dabryanus and 12 male Acipenser dabryanus were taken, where the Acipenser dabryanus were from Chinese Sturgeon Research Institute, China Three Gorges Corporation.

As shown in FIG. 2, it can be seen that all 12 female Acipenser dabryanus showed two bands, with sizes of 473 bp and 717 bp, respectively; all 12 male Acipenser dabryanus showed one band with a size of 717 bp. The results of this experiment were consistent with the results of genetic sex identification of Acipenser dabryanus obtained through spawning and sperm squeezing.

### Embodiment 3: Identification of Genetic Sex of Acipenser ruthenus

The present Embodiment 3 may refer to the steps of Embodiment 1, with the difference being that: after the genetic sex of Acipenser ruthenus was determined through dissection, fin strips from 12 female Acipenser ruthenus and 12 male Acipenser ruthenus were taken, where the Acipenser ruthenus were from Yichang Sturgeon Farm, Hubei, China.

As shown in FIG. 3, it can be seen that all 12 female Acipenser ruthenus showed two bands, with sizes of 473 bp and 717 bp, respectively; all 12 male Acipenser ruthenus showed one band with a size of 717 bp. The results of this experiment were consistent with the results of genetic sex identification of Acipenser ruthenus obtained through dissection.

### Embodiment 4: Identification of Genetic Sex of Acipenser schrenckii

The present Embodiment 4 may refer to the steps of Embodiment 1, with the difference being that: after the genetic sex of Acipenser schrenckii was determined through dissection, fin strips from 12 female Acipenser schrenckii and 12 male Acipenser schrenckii were taken, where the Acipenser schrenckii were from Sturgeon Farm, Yichang, Hubei, China.

As shown in FIG. 4, it can be seen that all 12 female Acipenser schrenckii showed two bands, with sizes of 473 bp and 717 bp, respectively; all 12 male Acipenser schrenckii showed one band with a size of 717 bp. The results of this experiment were consistent with the results of genetic sex identification of Acipenser schrenckii obtained through dissection.

### Embodiment 5: Identification of Genetic Sex of Acipenser schrenckii♂×Huso Dauricus♀

The present Embodiment 5 may refer to the steps of Embodiment 1, with the difference being that: the Acipenser schenckii♂×Huso dauricus♀ used in the present experiment was the first-generation offspring of hybridization with the Acipenser schrenckii as a male parent and the Huso dauricus as a female parent; after the genetic sex of Acipenser schrenckii♂×Huso dauricus♀ was determined through dissection, fin strips from 12 female Acipenser schrenckii♂×Huso dauricus♀ and 12 male Acipenser schrenckii♂×Huso dauricus♀ were taken, where the Acipenser schrenckii♂×Huso dauricus♀ were from Yichang Sturgeon Farm, Hubei, China.

As shown in FIG. 5, it can be seen that all 12 female Acipenser schrenckii♂×Huso dauricus♀ showed two bands, with sizes of 473 bp and 717 bp, respectively; all 12 male Acipenser schrenckii♂×Huso dauricus♀ showed one band with a size of 717 bp. The results of this experiment were consistent with the results of genetic sex identification of Acipenser schrenckii♂×Huso dauricus♀ obtained through dissection.

### Embodiment 6: Identification of Genetic Sex of Acipenser schrenckii♂×Acipenser Baerii♀

The present Embodiment 6 may refer to the steps of Embodiment 1, with the difference being that: the Acipenser schrenckii♂×Acipenser baerii? used in the present experiment was the first-generation offspring of hybridization with the female Acipenser baerii and the male Acipenser schrenckii; after the genetic sex of Acipenser schrenckii♂×Acipenser baerii♀ was determined through dissection, fin strips from 12 female Acipenser schrenckii♂×Acipenser baerii♀ and 12 male Acipenser schrenckii♂×Acipenser baerii♀ were taken, where the Acipenser schrenckii♂×Acipenser baerii♀ were from Yichang Sturgeon Farm, Hubei, China.

As shown in FIG. 6, it can be seen that all 12 female Acipenser schrenckii♂×Acipenser baerii♀ showed two bands, with sizes of 473 bp and 717 bp, respectively; all 12 male Acipenser schrenckii♂×Acipenser baerii♀ showed one band with a size of 717 bp. The results of this experiment were consistent with the results of genetic sex identification of Acipenser schrenckii♂×Acipenser baerii obtained through dissection.

### Embodiment 7: Identification of Genetic Sex of Acipenser baerii

The present Embodiment 7 may refer to the steps of Embodiment 1, with the difference being that: after the genetic sex of Acipenser baerii was determined through dissection, fin strips from 12 female Acipenser baerii and 12 male Acipenser baerii were taken, where the Acipenser baerii were from Yichang Sturgeon Farm, Hubei, China.

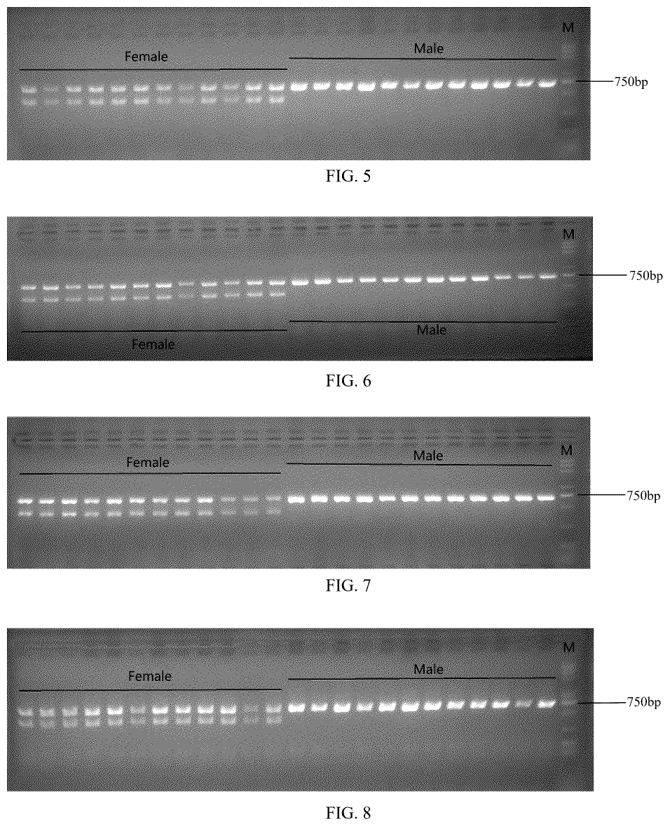

As shown in FIG. 7, it can be seen that all 12 female Acipenser baerii showed two bands, with sizes of 473 bp and 717 bp, respectively; all 12 male Acipenser baerii showed one band with a size of 717 bp. The results of this experiment were consistent with the results of genetic sex identification of Acipenser baerii obtained through dissection.

### Embodiment 8: Identification of Genetic Sex of Huso dauricus

The present Embodiment 8 may refer to the steps of Embodiment 1, with the difference being that: after the genetic sex of Huso dauricus was determined through dissection, fin strips from 12 female Huso dauricus and 12 male Huso dauricus were taken, where the Huso dauricus were from Sturgeon Farm, Beijing, China.

As shown in FIG. 8, it can be seen that all 12 female Huso dauricus showed two bands, with sizes of 473 bp and 717 bp, respectively; all 12 male Huso dauricus showed one band with a size of 717 bp. The results of this experiment were consistent with the results of genetic sex identification of Huso dauricus obtained through dissection.

### Embodiment 9: Identification of Genetic Sex of Acipenser gueldenstaedti

The present Embodiment 9 may refer to the steps of Embodiment 1, with the difference being that: after the genetic sex of Acipenser gueldenstaedti was determined through dissection, fin strips from 12 female Acipenser gueldenstaedti and 12 male Acipenser gueldenstaedti were taken, where the Acipenser gueldenstaedti were from Sturgeon Farm, Beijing, China.

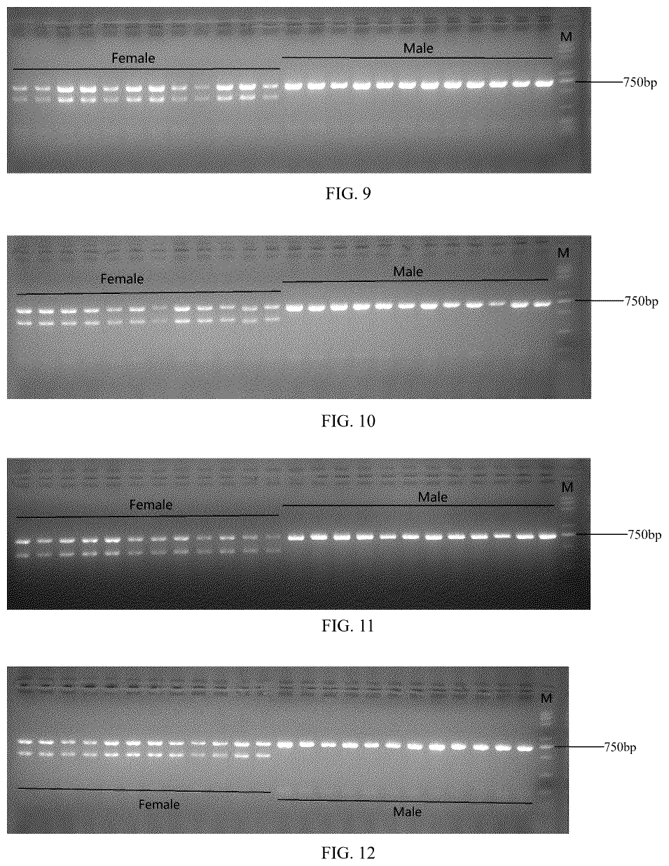

As shown in FIG. 9, it can be seen that all 12 female Acipenser gueldenstaedti showed two bands, with sizes of 473 bp and 717 bp, respectively; all 12 male Acipenser gueldenstaedti showed one band with a size of 717 bp. The results of this experiment were consistent with the results of genetic sex identification of Acipenser gueldenstaedti obtained through dissection.

### Embodiment 10: Identification of Genetic Sex of Acipenser stellatus

The present Embodiment 10 may refer to the steps of Embodiment 1, with the difference being that: after the genetic sex of Acipenser stellatus was determined through dissection, fin strips from 12 female Acipenser stellatus and 12 male Acipenser stellatus were taken, where the Acipenser stellatus were from Sturgeon Farm, Beijing, China.

As shown in FIG. 10, it can be seen that all 12 female Acipenser stellatus showed two bands, with sizes of 473 bp and 717 bp, respectively; all 12 male Acipenser stellatus showed one band with a size of 717 bp. The results of this experiment were consistent with the results of genetic sex identification of Acipenser stellatus obtained through dissection.

### Embodiment 11: Identification of Genetic Sex of Huso huso

The present Embodiment 11 may refer to the steps of Embodiment 1, with the difference being that: after the genetic sex of Huso huso was determined through an endoscopic technique, fin strips from 12 female Huso huso and 12 male Huso huso were taken, where the Huso huso were from Sturgeon Farm, Zhejiang, China.

As shown in FIG. 11, it can be seen that all 12 female Huso huso showed two bands, with sizes of 473 bp and 717 bp, respectively; all 12 male Huso huso showed one band with a size of 717 bp. The results of this experiment were consistent with the results of genetic sex identification of Huso huso obtained through the endoscopic technique.

### Embodiment 12: Identification of Genetic Sex of Acipenser fulvescens

The present Embodiment 12 may refer to the steps of Embodiment 1, with the difference being that: after the genetic sex of Acipenser fulvescens was determined through dissection, fin strips from 12 female Acipenser fulvescens and 12 male Acipenser fulvescens were taken, where the Acipenser fulvescens were from Sturgeon Farm, Xinjiang, China.

As shown in FIG. 12, it can be seen that all 12 female Acipenser fulvescens showed two bands, with sizes of 473 bp and 717 bp, respectively; all 12 male Acipenser fulvescens showed one band with a size of 717 bp. The results of this experiment were consistent with the results of genetic sex identification of Acipenser fulvescens obtained through dissection.

### Embodiment 13: Identification of Genetic Sex of Acipenser nudiventris

The present Embodiment 13 may refer to the steps of Embodiment 1, with the difference being that: after the genetic sex of Acipenser nudiventris was determined through dissection, fin strips from 12 female Acipenser nudiventris and 12 male Acipenser nudiventris were taken, where the Acipenser nudiventris were from Sturgeon Farm, Xinjiang, China.

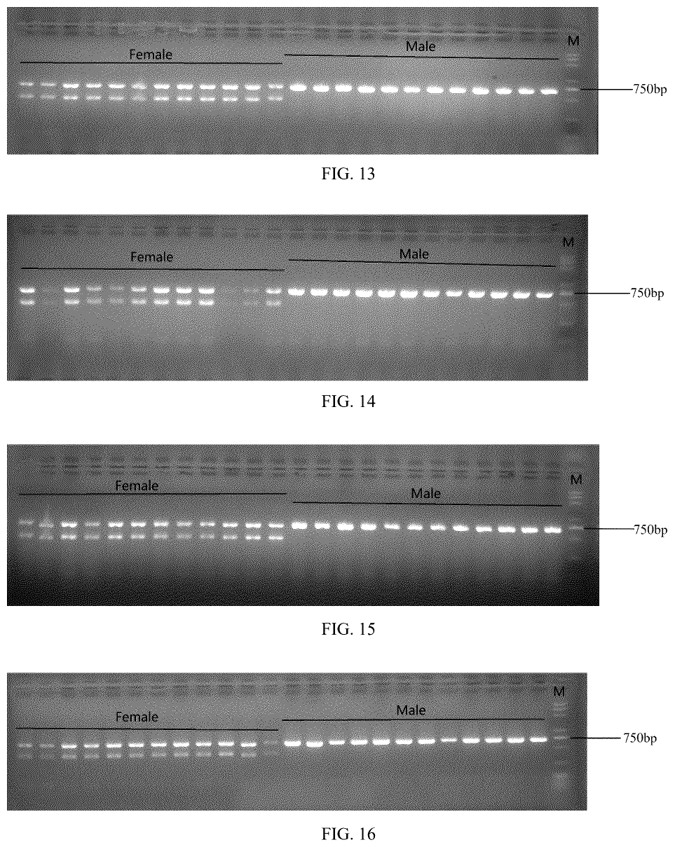

As shown in FIG. 13, it can be seen that all 12 female Acipenser nudiventris showed two bands, with sizes of 473 bp and 717 bp, respectively; all 12 male Acipenser nudiventris showed one band with a size of 717 bp. The results of this experiment were consistent with the results of genetic sex identification of Acipenser nudiventris obtained through dissection.

### Embodiment 14: Identification of Genetic Sex of Acipenser persicus

The present Embodiment 14 may refer to the steps of Embodiment 1, with the difference being that: after the genetic sex of Acipenser persicus was determined through dissection, fin strips from 12 female Acipenser persicus and 12 male Acipenser persicus were taken, where the Acipenser persicus were from Sturgeon Farm, Xinjiang, China.

As shown in FIG. 14, it can be seen that all 12 female Acipenser persicus showed two bands, with sizes of 473 bp and 717 bp, respectively; all 12 male Acipenser persicus showed one band with a size of 717 bp. The results of this experiment were consistent with the results of genetic sex identification of Acipenser persicus obtained through dissection.

### Embodiment 15: Identification of Genetic Sex of Acipenser transmontanus

The present Embodiment 15 may refer to the steps of Embodiment 1, with the difference being that: after the genetic sex of Acipenser transmontanus was determined through dissection, fin strips from 12 female Acipenser transmontanus and 12 male Acipenser transmontanus were taken, where the Acipenser transmontanus were from Sturgeon Farm, Xinjiang, China.

As shown in FIG. 15, it can be seen that all 12 female Acipenser transmontanus showed two bands, with sizes of 473 bp and 717 bp, respectively; all 12 male Acipenser transmontanus showed one band with a size of 717 bp. The results of this experiment were consistent with the results of genetic sex identification of Acipenser transmontanus obtained through dissection.

### Embodiment 16: Identification of Genetic Sex of Acipenser medirostris

The present Embodiment 16 may refer to the steps of Embodiment 1, with the difference being that: after the genetic sex of Acipenser medirostris was determined through dissection, fin strips from 12 female Acipenser medirostris and 12 male Acipenser medirostris were taken, where the Acipenser medirostris were from Sturgeon Farm, Xinjiang, China.

As shown in FIG. 16, it can be seen that all 12 female Acipenser medirostris showed two bands, with sizes of 473 bp and 717 bp, respectively; all 12 male Acipenser medirostris showed one band with a size of 717 bp. The results of this experiment were consistent with the results of genetic sex identification of Acipenser medirostris obtained through dissection.

In summary, the present application is applicable to various sturgeon species and has good universality. The genetic sex of sturgeon identified in the present application is consistent with that obtained by the dissection method, the spawning and sperm squeezing method, or the endoscopic technique, indicating that the present application can correctly identify the genetic sex of sturgeon. At the same time, compared with the dissection method, the present application only requires extraction of trace amount of DNA from the sturgeon to be detected, which will not cause significant impact or damage to the sturgeon; compared with the spawning and sperm squeezing method and the endoscopic technique, the present application is not limited by the age of sturgeon and can perform the identification of genetic sex throughout the entire life cycle of sturgeon. Due to the primer combination of the present application providing two pairs of primers, it can also avoid experimental errors caused by unsuccessful DNA extraction, wrong addition of drugs or omission of drugs, ensuring the accuracy of sturgeon sex identification. In addition, because the two DNA fragments amplified by two pairs of primers are located on the same chromosome with a distance between them of no more than 20 kbp, the experimental errors caused by severe degradation of DNA can be effectively reduced. The present application can provide assistance for sex-controlled breeding of sturgeon and protection and utilization of germplasm resources, as well as technical support for biodiversity research and the great conservation work of Yangtze River.

Finally, it should be noted that the above embodiments are only used to illustrate the technical solutions of the present application, but not to limit them. Although the present application has been described in detail with reference to the aforementioned embodiments, it should be understood by persons skilled in the art that the technical solutions recorded in the aforementioned embodiments may still be modified or some or all of the technical features of the technical solutions may be equivalently substituted; while these modifications or substitutions do not make the essence of the corresponding technical solutions depart from the scope of the technical solutions of various embodiments of the present application.

What is claimed is:

1. A primer combination for identifying genetic sex of sturgeon, comprising a first primer pair and a second primer pair, wherein the first primer pair comprises a first primer and a second primer, and a nucleotide sequence of the first primer is shown in SEQ ID NO: 1 and a nucleotide sequence of the second primer is shown in SEQ ID NO: 2; and the second primer pair comprises a third primer and a fourth primer, and a nucleotide sequence of the third primer is shown in SEQ ID NO: 3 and a nucleotide sequence of the fourth primer is shown in SEQ ID NO: 4.

2. The primer combination according to claim 1, wherein the first primer pair is used for amplifying a specific DNA fragment of a female sturgeon, and the specific DNA fragment of the female sturgeon has a nucleotide sequence shown in SEQ ID NO: 5 and a fragment size of 473 bp; and the second primer pair is used for amplifying a DNA fragment shared by the female sturgeon and a male sturgeon, and the DNA fragment shared by the female sturgeon and the male sturgeon has a nucleotide sequence shown in SEQ ID NO: 6 and a fragment size of 717 bp.

3. The primer combination according to claim 2, wherein the sturgeon comprises at least one of Acipenser sinensis, Acipenser dabryanus, Acipenser ruthenus, Acipenser schrenckii, Acipenser schrenckii♂×Huso dauricus♀, Acipenser schrenckii♂×Acipenser baerii♀, Acipenser baerii, Huso dauricus, Acipenser gueldenstaedti, Acipenser stellatus, Huso huso, Acipenser fulvescens, Acipenser nudiventris, Acipenser persicus, Acipenser transmontanus, or Acipenser medirostris.

4. The primer combination according to claim 1, wherein the sturgeon comprises at least one of Acipenser sinensis, Acipenser dabryanus, Acipenser ruthenus, Acipenser schrenckii, Acipenser schrenckii♂×Huso dauricus♀, Acipenser schrenckii♂×Acipenser baerii♀, Acipenser baerii, Huso dauricus, Acipenser gueldenstaedti, Acipenser stellatus, Huso huso, Acipenser fulvescens, Acipenser nudiventris, Acipenser persicus, Acipenser transmontanus, or Acipenser medirostris.

5. A kit for identifying genetic sex of sturgeon, comprising the primer combination according to claim 1.

6. The kit according to claim 5, wherein the first primer pair is used for amplifying a specific DNA fragment of a female sturgeon, and the specific DNA fragment of the female sturgeon has a nucleotide sequence shown in SEQ ID NO: 5 and a fragment size of 473 bp; and the second primer pair is used for amplifying a DNA fragment shared by the female sturgeon and a male sturgeon, and the DNA fragment shared by the female sturgeon and the male sturgeon has a nucleotide sequence shown in SEQ ID NO: 6 and a fragment size of 717 bp.

7. The kit according to claim 5, wherein the sturgeon comprises at least one of Acipenser sinensis, Acipenser dabryanus, Acipenser ruthenus, Acipenser schrenckii, Acipenser schrenckii♂×Huso dauricus♀, Acipenser schrenckii♂×Acipenser baerii♀, Acipenser baerii, Huso dauricus, Acipenser gueldenstaedti, Acipenser stellatus, Huso huso, Acipenser fulvescens, Acipenser nudiventris, Acipenser persicus, Acipenser transmontanus, or Acipenser medirostris.

8. The kit according to claim 5, further comprising at least one of water, PCR buffer, dNTPs, DNA polymerase, or loading buffer.

9. A method for identifying genetic sex of sturgeon, comprising the following steps:
extracting DNA of a sturgeon sample to be detected;
performing a PCR amplification using the primer combination according to claim 1 with the DNA of the sturgeon sample to be detected as a template to obtain a PCR amplification product; and
performing an agarose gel electrophoresis detection on the PCR amplification product, and identifying the genetic sex of sturgeon as female when two bands of 473 bp and 717 bp appear in a detection result and identifying the genetic sex of sturgeon as male when one band of 717 bp appears in the detection result.

10. The method according to claim 9, wherein the sturgeon sample to be detected comprises at least one of fin strip of the sturgeon or mucus of the sturgeon.

11. The method according to claim 9, wherein in the PCR amplification, a PCR reaction system comprises 3-5 μL of the DNA template, 0.5-1.5 μL of the first primer, 0.5-1.5 μL of the second primer, 0.25-0.75 μL of the third primer, 0.25-0.75 μL of the fourth primer, 10-15 μL of 2×Taq Master Mix, and 4-6 μL of ddH₂O.

12. The method according to claim 9, wherein in the PCR amplification, a PCR amplification program comprises pre-denaturation at 93-95° C. for 3 minutes; denaturation at 93-95° C. for 10 seconds, renaturation at 54-56° C. for 10 seconds, and extension at 72° C. for 19 seconds, for a total of 35 cycles; and finally extension at 72° C. for 10 minutes.

13. A method for identifying genetic sex of sturgeon, comprising the following steps:
extracting DNA of a sturgeon sample to be detected;
performing a PCR amplification using the kit according to claim 5 with the DNA of the sturgeon sample to be detected as a template to obtain a PCR amplification product; and
performing an agarose gel electrophoresis detection on the PCR amplification product, and identifying the genetic sex of sturgeon as female when two bands of 473 bp and 717 bp appear in a detection result and identifying the genetic sex of sturgeon as male when one band of 717 bp appears in the detection result.

14. The method according to claim 13, wherein the sturgeon sample to be detected comprises at least one of a fin strip of the sturgeon or a mucus of the sturgeon.

15. The method according to claim 13, wherein in the PCR amplification, a PCR reaction system comprises 3-5 μL of the DNA template, 0.5-1.5 μL of the first primer, 0.5-1.5 μL of the second primer, 0.25-0.75 μL of the third primer, 0.25-0.75 μL of the fourth primer, 10-15 μL of 2×Taq Master Mix, and 4-6 μL of ddH₂O.

16. The method according to claim 13, wherein in the PCR amplification, a PCR amplification program comprises pre-denaturation at 93-95° C. for 3 minutes; denaturation at 93-95° C. for 10 seconds, renaturation at 54-56° C. for 10 seconds, and extension at 72° C. for 19 seconds, for a total of 35 cycles; and finally extension at 72° C. for 10 minutes.


SEQUENCE LISTING (6 sequences)

SEQ ID NO: 1 - DNA, 16 bp, synthetic construct
  source 1..16: mol_type = other DNA
ttcgggcaggacaact

SEQ ID NO: 2 - DNA, 16 bp, synthetic construct
  source 1..16: mol_type = other DNA
aacaatcgggcaacca

SEQ ID NO: 3 - DNA, 18 bp, synthetic construct
  source 1..18: mol_type = other DNA
ggattgaggaggatgtgc

SEQ ID NO: 4 - DNA, 18 bp, synthetic construct
  source 1..18: mol_type = other DNA
gtgaaataggggactggc

SEQ ID NO: 5 - DNA, 473 bp, Sturgeon ichtadenovirus A
  source 1..473: mol_type = genomic DNA
ttcgggcaggacaactggacatctgaagaaaatcccttttcctgaggccaggttgacttggtgaatatacgaaagattacagtattttatgaagtagaaacaacctagatgtatgtgccaaaagaggaaatgggttgcatttatggttaggaacagactgtctctaaacattacagcagacagttaaccgtctgtgtgccagacatgagtattttattacggtgagtatacgaaaaattacagtatttttgcactatacgaaagattacagtattttagcagtcatttgtaataaaataatcactgaagggagtgttttacaaccagtgcacggctataaagactgcccagagcctcatgtctcatatagagacttgcgtaaaaaattacaagaacagaaaaaacaatgaaggggtcagacatcggataaggtggcatatatacttagagattcttttggttgcccgattgtt

SEQ ID NO: 6 - DNA, 717 bp, Sturgeon ichtadenovirus A
  source 1..717: mol_type = genomic DNA
gtgaaataggggactggctgcagctggaacctgctgccaaaaccaggggtgtcccatcagccctctggcacactatgttgaacagtagataaaactttccagtcagcagcaagcttgctctgcaagccccagcaagccccagcagctaggctggattactaaatgcctttccacctgccgtcactacagtgtacttcccccatctccctgaaggttagattgctgctgggagttatggtgtctgcctgaccctgggccctattgctgggtttgttccaaaggtttgctgaatgaaaaaaatgtgtgctaggctgcacattgtgtactggattatatacacaacagcaacctgctgcagtctccactgcagctattcccttcaaggcaaatctttttccctctcgctctgtgctgtgggatgcccagactgaccaccttgaggctgaatggcagcagaaagaaagaaaattagtcatttgttttataggcagcctaagctgggtttgttgcattctcgaaatgaaaagcaagtgaactgtgaggtatttaaacaagcgtataacgttcgttttttttttcttaattttttcaaatgcacagcaaaataagggaccgttatacatggtgtgttcattctatttatcataacacctgaatttgccagaagcaatcacaatgaaagagccacttgctctgaaagcacatcctcctcaatcc